(19) United States
(12) Patent Application Publication
(10) Pub. No.: US 2019/0059752 A1
(43) Pub. Date: Feb. 28, 2019

(54) Method and apparatus for cuff less blood pressure monitoring based on simultaneously measured ECG and PPG signals designed in wristband form for continuous wearing

(71) Applicant: PLANEXTA, INC., Santa Monica, CA (US)

(72) Inventors: Tetiana BOTSVA, Dnipro (GB); Dmytro CHERNETCHENKO, Dnipro (GB)

(21) Appl. No.: 15/687,678

(22) Filed: Aug. 28, 2017

(51) Int. Cl. A61B 5/021; A61B 5/0404; A61B 5/0408; A61B 5/0456; A61B 5/1455; A61B 5/00; A61B 5/0205

(52) U.S. Cl. CPC A61B 5/02125; A61B 5/02427; A61B 5/0404; A61B 5/04085; A61B 5/0456; A61B 5/14552; A61B 5/681; A61B 5/6843; A61B 5/7221; A61B 5/0006; A61B 5/7475; A61B 5/0205; A61B 2560/0223; A61B 2562/0209; A61B 2562/0238; A61B 2560/0214; A61B 2562/066; A61B 5/02116

(57) ABSTRACT

The present invention relates to the method and apparatus for cuffless measuring of blood pressure value from one limb of a subject. Proposed herein method of measuring blood pressure based on pressure calculation method from pulse wave velocity (PWV) value, which does not require any external device for calibration. PWV is calculated from three main signals: the heart electrical activity signal (ECG) and two photoplethysmographic signals (PPG). The described herein device comprises a set of ECG electrodes, optical and electronic sensors, a microcontroller for signal processing, a power supply unit, user interface means, and a wireless communication unit. The device is designed to monitor blood pressure and can be used for medical purposes or self-monitoring in everyday life.

### FIELD OF THE INVENTION

The present invention relates to methods and apparatus for measuring a blood pressure value from single extremity of a subject, and more particularly to methods based on calculating pressure from the pulse wave velocity value measured between two points on an extremity. In addition, the present invention relates to devices for body state (condition) diagnosing and can be used both for medical purposes and self-monitoring in daily life.

### DESCRIPTION OF THE RELATED ART

It is well known that arterial pressure is one of the main parameters in determining the state of the human body, which is why its continuous (regular) monitoring is so important. The possibility of using a wearable device for regular measurement of blood pressure simplifies the patient's health monitoring process and improves the medicare quality, and can be used, for example, to monitor chronic hypertension or a recovery process. But the realization true non-invasive measurement of blood pressure (NIBP) is associated with a number of problems, for example, taking into account parameters characterizing the state of the artery at the measurement site (diameter, artery wall stiffness and tension, etc.). Ideally, a wearable blood pressure measuring device should be non-invasive, lightweight, easy to use, ergonomic akin wristwatch. However, the presently existing methods and devices for non-invasive blood pressure measurement, described below, completely or partially do not meet these requirements.

The oscillometric measuring NIBP is the most common method and is used both in clinical settings and in everyday life. This method is based on the following principle: when the external pressure of the cuff is equal to the internal arterial pressure, the magnitude of arterial volume pulsations is maximal because of the mechanical properties of arteries. Oscillometric measurement devices typically use an electronic pressure sensor with a numerical readout of blood pressure. In most cases the cuff is inflated and released by an electrically operated pump and valve, which may be fitted on the wrist (elevated to heart height), although the upper arm position is preferred. Initially the cuff is inflated to a pressure in excess of the systolic arterial pressure, and then the pressure gradually reduces to below diastolic pressure. Once the blood flow is present, but restricted, the cuff pressure will vary periodically in synchrony with the cyclic expansion and contraction of the brachial artery. The values of systolic and diastolic pressure are computed from the raw data, using an algorithm. Although the apparatuses using this method are standard in health care, they nevertheless have a number of disadvantages: (i) the cuff creating circumferential compression of the limb causes discomfort and can cause bruising and disturbance (interruption) of sleep, when measurements are taken during sleep; (ii) the device output values are very susceptible to movement, cause the motion artifact can not be distinguished from the pulsation of the arterial volume; (iii) such devices are difficult to miniaturize, so often they are quite large in size and weight; (iv) in general, the accuracy of their measurements with relatively to the invasive method is 5-8 mmHg, according to “Clinical Practice Guideline: Non-Invasive Blood Pressure Measurement with Automated Devices.” Full Version “by Susan Barnason et al. (6.6-9.7 mmHg according to “Accuracy of the oscillometric blood pressure measurement according to the relation between cuff size and upper-arm circumference in critically ill patients.” By Bur A. I., Hirschl M. M. et al.) More convenient versions of the oscillometric cuff are fixed to the wrist, however they have a large measurement error, especially in cases (v) when the wrist is not at the heart level during the measurement, (vi) if the patient has distal arterial occlusions (atherosclerosis) or vasospasm, (vii) the error increases with the age of the patient (after 40 years), which is associated with age-related changes in blood vessels.

Another method of measuring NIBP is The Volume Clamp Method, the brightest representatives using this method are the Portapres®/Finometer® device by Finapres Medical Systems, which allows non-invasive measurement of the blood pressure waveform from the finger using photoplethysmographic (PPG) technology. Data from the photodetector (the intensity of the light flux passed through the patient's body) often in the red and infrared spectrum are transformed into a level of oxygen saturation and a time-dependent waveform called a photoplethysmogram (PPG), which indicates both the pulse rate and the volume change in the optical density of the underlying artery, caused by the spread of pressure pulse. This method is based on the regularity: if the PPG signal does not change, then the value of the arterial transmural pressure does not change, and vice versa. In other words, if the PPG signal is flat, the transmural pressure waveform is flat too. Using an extremely fast servo system with an actuator for the finger cuff, Finapres adjusts the pressure in the cuff to maintain the reference PPG signal unchanged (flat) for all systoles and diastoles during measurement. Thus, the method is known as “the volume clamp”, since the volume of blood in the finger is kept constant. The waveform of the cuff pressure which is necessary to maintain a flat PPG signal is equal and opposite to the digital value of the blood pressure. The main advantage of this method and device is the ability to continuously measure the blood pressure waveform, as well as the presence of a correction mechanism for the error of the hydrostatic displacement (relative to the heart). Nevertheless, it has a number of disadvantages: (i) for long measurements, the user feels discomfort, in this connection the device has two cuffs and measurements are made alternately from different fingers; (ii) the operating of the pneumatic cuff requires a bulky, power-thirsty actuation system that makes the device miniaturization quite problematic; (iii) the accuracy of measurements decreases with time, due to the adaptation of the vessels to external influences.

Volume plethysmography (for example, a wrist sensor by Empirical Technologies). Devices using this method can be miniature, lightweight and low-power. However, their main drawback is that the plethysmograph signal is a volumetric signal that is very different from the pressure signal. The pulsation volume is a complex function of blood pressure, mechanical properties of arteries and pressure of the sensor itself.

The most promising at the moment is the method of measuring blood pressure by the pulse transition time (PTT) or the pulse wave velocity (PWV). The pulse wave velocity (PWV) is the rate at which the pulse propagates through the arterial tree, and it can be calculated from the pulse transition time (PTT), which is defined as the passage time of pulse produced by cardiac contractions along the human arterial system between two measuring sites. And as many studies show, these parameters correlate well with the values of both systolic and diastolic pressures. Typically, the value of PTT is determined using an electrocardiograph (ECG), which electrodes are often attached to the patient's chest, and a pulse oximeter, a sensor for detecting blood oxygenation (SpO2), recording their signals in parallel and determining the time delay between the corresponding wave points of both signals. From the time-dependent ECG signal identify specific sharp peaks called the “QRS complex,” which indicates the onset of depolarization of the ventricles of the heart and indirectly indicates the moment of contraction of the heart and the beginning of the subsequent pressure wave. The SpO2 device most often has the form of a band or clothespins fixed on the finger, wrist or ear of the patient and containing an optical system operating in both the red and the infrared spectral range.

Usually, PTT is defined as the time separating the maximum of the QRS complex (the beginning of cardiac ventricular depolarization) and the minimum or the beginning of the positive wave of the optical signal (the start of the pressure pulse). PTT primarily depends on the elasticity of the arteries, the distance that the pressure wave passes (approximately defined as the arm length) and blood pressure. All individual parameters influencing blood pressure can be described as certain coefficients. To calibrate and remove the pressure values' dependence on individual parameters, a common used cuff is fixed on the patients arm for one or more calibration measurements, and then removed. PTT is usually inversely proportional to blood pressure, that is, an increase in PTT indicates a decrease in blood pressure.

A number of granted U.S. patents describe the relationship between PTT and blood pressure. For example, U.S. Pat. Nos. 5,316,008; 5,857,975; 5,865,755; and 5,649,543 each describe an apparatus that includes conventional sensors that measure an ECG and optical waveform, which are then processed to determine PTT.

One representative of the devices using this method is the ViSi Mobile device by Sotera Wireless, described in U.S. Pat. No. 8,200,321 B2 and the brochure “ViSi Mobile. Surveillance Monitoring System”. As a standard, the device with a sensor on the finger is designed for continuous measurement of saturation and heart rate. The optional addition of a chest sensor system enables the measurement of respiratory rate, body temperature, ECG signal and body position/movement monitoring. The arterial pressure is calculated from the pulse transition time (PTT or PAT) determined from ECG and PPG waveforms. The PPG signal is readied with the ViSi Mobile device, fixed to the wrist with the thumb saturation sensor. The ECG signal is recorded using a system of chest sensors attached to the patient's skin and connected to the ViSi Mobile unit on the wrist by wires. ViSi Mobile's proprietary cNIBP is based on Pulse Arrival Time (PAT). After cuff-based calibration, and cuff is removed, blood pressure is measured based on beat-to-beat blood pressure. The main disadvantages of this device are the need to use a compression cuff for calibration, and the use of chest electrodes to record the ECG signal, which can cause discomfort and restrict the freedom of patient's movement.

### SUMMARY OF THE INVENTION

In accordance with one aspect of the present invention, as a method to achieve this objective was chosen a method of indirect determining the systolic and diastolic pressure via pulse wave velocity (PWV), which the European Society of Hypertension recognized as an important parameter in the study of hypertension (according to “2013 ESH/ESC Guidelines for the management of arterial hypertension” by: Mancia G., Fagard R., Narkiewicz K. et al.). Pulse wave velocity (PVW) through arterial tree is calculated from the two main information ECG and PPG signals, as the length of the pulse wave propagation path divided by the pulse transition time (PTT) of this distance. The PTT equals to a time delay between the corresponding points on the ECG and PPG signals (or two PPG signals recorded from two spaced apart points). The main advantage of this method over existing analogs is the way of calibration without using a compression cuff. In addition, the present invention using the method of recording an ECG signal from one limb, allows blood pressure measurements to be made by a device in which all sensors are assembled in single housing and fixed to the user's wrist, unlike the nearest analog VISI Mobile by Sotera Wireless.

This method includes the following steps: parallel registration of ECG and PPG signals; detection of the R-peak on the ECG signal and the maximum of PPG signals corresponding to the same heart beat; PWV and PTT calculation; converting of these parameters into the value of systolic and diastolic pressure by a special algorithm; recording of the received data for visualization, the subsequent processing and transfer on external devices. Preliminary calibration measurements allow to reduce the influence of individual physiological parameters of the user on the result of measurement.

According to another aspect of the invention, there is provided an electronic device comprising: a block of sensors, including at least a set (kit) of two ECG electrodes and two PPG photosensors representing the main information sensors, as well as a set of sensors necessary for calibration—an accelerometer and a barometer; analog filtering and signal amplification unit comprising an automatic gain control system; microcontroller unit, where the signal is digitized and subjected to further processing, during which all parameters and output data are calculated. The electronic device further comprises: a memory unit for storing received data; wireless communication module for data transfer to a mobile device; an interface block containing user buttons and a screen for visualizing the output data; battery for autonomous operation and charge control unit.

As a third aspect of the present invention, an algorithm for the operation of the electronic device described herein, and in particular an algorithm for calculating systolic and diastolic pressure values, is provided. The algorithm for performing calibration measurements is presented in this invention as an auxiliary process to reduce the dependence of the final results on the individual parameters of the subject.

The method and the electronic device according to the invention provide several advantages. The main and most important advantage is the possibility of non-invasive measurement of blood pressure without the use of a compression cuff. Also, unlike the closest analogue of VISI Mobile by Sotera Wireless, all the sensors are located in the single housing in size comparable to the wristwatch and fixed to the wrist, specifically the ECG signal is recorded not from the chest electrodes but from one hand.

Recording of the ECG signal from one limb of the subject with the help of dry electrodes and detection of R-peaks in the recorded signal is implemented by methods referred to herein as “ECG signal recording method” and “R-peaks detection method”. These methods are carried out with a body-worn device fixed on the user's wrist and allows to record the ECG signal in conjunction with other signals, calculate vital signs, save them and transfer them to external mobile devices. The methods of ECG recording and R-peaks detection are described in detail in the co-pending patent application entitled: THE METHOD OF REGISTERING THE INTERVALS BETWEEN ADJACENT R-PEAKS OF ECG SIGNAL WITH ONE HAND IN ORDER TO DIAGNOSE AND ASSESS THE STATE OF THE HUMAN BODY AND HEART RATE VARIABILITY WEARABLE MONITORING DEVICE (U.S. Ser. No. 15/442,631; filed Mar. 18, 2017), the contents of which are fully incorporated herein by reference.

### BRIEF DESCRIPTION OF THE DRAWINGS

Essence and advantages of the present invention are seen in the following detailed description in conjunction with the attached drawings. These drawings are intended to provide a better understanding of the present invention, but they are in no way intended to limit the scope of the invention. It should be remembered that the various embodiments of the present invention are not limited to the precise arrangements and instrumentalities shown in the drawings.

In the Drawings:

FIG. 1a shows the front side of the electronic device with means of housing fixing and interface;

FIG. 1b shows the housing backside of the device with locations of electrodes and holes for photo detectors;

FIG. 2a illustrates detectors unit of block-diagram of device;

FIG. 2b is a part of block-diagram corresponding to interface of device;

FIG. 2c is a power management part of block-diagram;

FIG. 3a, 3b show algorithm of embedded software, specifically processes of preliminary preparation and measurements;

FIG. 3c shows part of embedded software algorithm corresponding to menu of device modes;

FIG. 4a shows ECG and 2 PPG signals recorded at the same time;

FIG. 4b shows the first derivative of the PPG signal with respect to time (d(PPG)/dt) in comparison with a PPG signal;

FIG. 5 shows how user should move during calibration measurement;

FIG. 6 is the calibration process algorithm;

FIG. 7 explains process of Blood Pressure measurement;

FIG. 8 discloses the principle of PPG detector operation;

FIG. 9 is a schematic representation of an artery section on which blood pressure measurements are taken;

FIG. 10a shows the artery compliance which is the curvilinear relationship between arterial pressure and blood volume;

FIG. 10b is dependence of the pulse transition time on the transmural pressure;

FIG. 10c shows the relationship between pulse wave velocity and transmural pressure.

### DETAILED DESCRIPTION OF THE PREFERRED EMBODIMENT

The electronic device described in this invention is assembled in a housing 102 and fixed to the user's hand by a strap 101. There is the visualization mean 103 on the front panel of the case, which is arranged in the form of a screen and the buttons 104, as a means of controlling the operation of the device. On the backside of the housing there are shown location of ECG electrodes 105, photodetector #1 106 and photodetector #2 107 (PPG #1 and PPG #2 respectively), the distance between which should be at least 20 mm. Since the electrodes must have direct and good contact with the skin, they have a rounded shape, which is necessary in order to minimize the discomfort caused to the user by continuous wearing. In the present embodiment, the photosensors do not have direct contact with the skin.

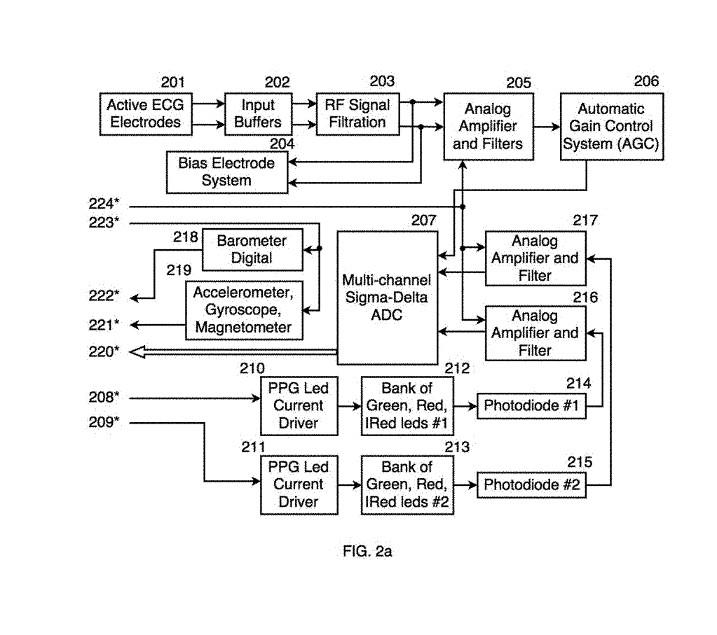

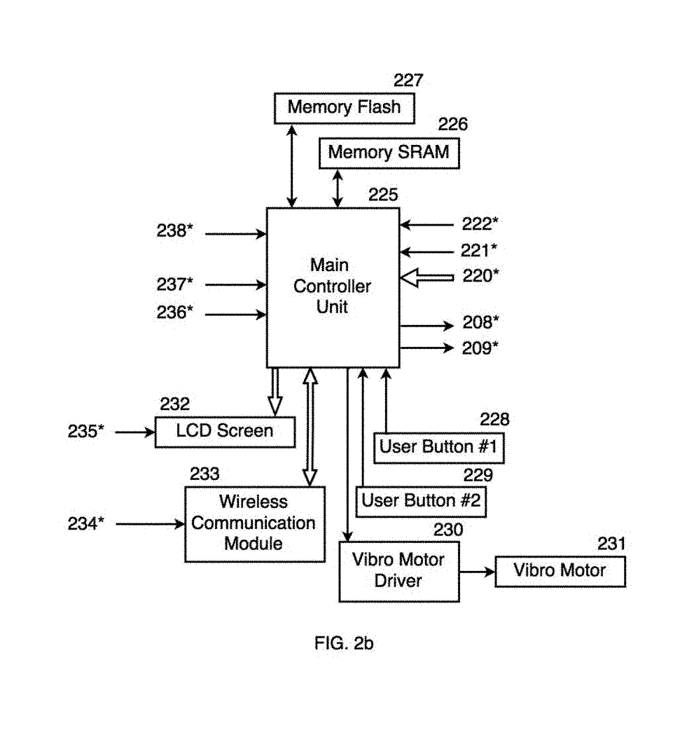

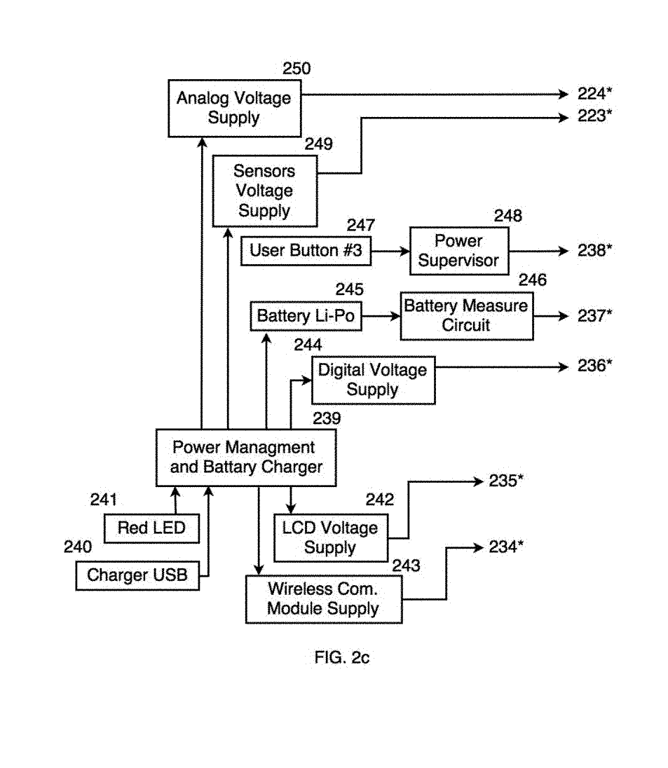

The block diagram of the device, explaining its internal structure and operating principle, is shown in FIG. 2a, 2b, 2c block by block. The electrodes record the minor fluctuations of the electric field generated by the human heart with an amplitude of about 0.5-1.5 mV. The potential from active electrodes 201 is transmitted through a cascade of buffer amplifiers 202 and RFI 203 to a differential instrumentation amplifier analog signal 205, that executes amplification potential difference between the two active electrodes 201, subsequent block 205 of the tract performs the primary filtering and noise reduction of the received signal, and sets the working frequency domain of the recorded signal in the range from 0.5 to 100 Hz. Fixed gain of the path is 100. To set the amplifier linearity, further suppress phase noise and dynamic range of potentials applied technology relative “reference” electrode, implemented in block 204. Full analog path gain factor is adjusted by the AGC (automatic gain control) with digital control 206. The gain of the programmable amplifier can assume the values 1, 2, 4, 5, 8, 10, 16, 32. As a result, the minimum gain of the analog tract is 100, and the maximum is 3200.

Each of the two photosensors includes a set of green, red and infrared LEDs 212, 213, as well as a photodiode of 214, 215. Using as a PPG sensor such a LEDs kit with different wavelengths makes it possible to realize the measuring not only the pulse rate, but also the degree of oxygen saturation of the blood—SpO2, as an additional function of this device. The brightness, and hence the luminous flux, of the LEDs is controlled by limiting the current supplied to the LEDs, which is controlled by two current drivers 210, 211 separately for each PPG sensor, which in turn are controlled by the main microcontroller 225, the control signals to which is transmitted via the channels 208*, 209*. The digitized signal from the ADC output via the information channel 220* is transmitted to the input of the microcontroller 225, where it is further processed.

In addition to ECG and PPG, the device has a number of additional sensors, specifically a digital barometer 218 and a block of accelerometer, gyroscope and magnetometer sensors 219. These sensors are designed to provide additional functionality to the device, and the accelerometer outputs data is involved in the calculation of blood pressure. The digital signal from the output of these sensors is transferred to the corresponding digital inputs 221*, 222* of the main microcontroller 225 for further processing.

The measurements' results of all parameters, as well as calculated data, the parameters values necessary for these calculations, their algorithm are stored in memory blocks: Flash memory 227 and SRAM memory 226. The microcontroller 225 records and reads data from the memory. The operating mode of the device (pressure measurement, calibration, barometer readings and other additional functions) is selected by pressing the user buttons 228, 229 in a certain sequence that form part of the user interface and provide menu management of operation of the device. To ensure communication between the device and the user, especially necessary information output, the device provides two means of information display: the LCD screen 232 and the vibro motor 231. The vibro motor signal is a short vibration and serves as a warning to the user, as a reminder that it is necessary to stop the hand (limb) movement. Signaling vibration is used for notification to user about start of the measurement process, as well as during the calibration process and in a number of other functions. The vibro motor is controlled by the main microcontroller 225 using the driver 230. LCD screen 232 is intended primarily for displaying measurement results, displaying menu items for operating modes of the device, and also for outputting special messages, such as error messages or warnings. To connect the device to an external mobile device, there is provided a wireless communication unit 233, which, if it is necessary, communicates and receives/transmits information with the mobile device.

The power supply of the device is provided by an external lithium polymer battery 245 Upt=3.7 V, Fault=300 mA/h. The battery is connected to the device PCB. To control the battery charge, a microcontroller unit 225 is used, as well as a battery power measurement circuit 246 that operates on the “divider” principle. Power management of the entire device and the battery charge is provided by block 239. The stabilized voltage for all components of the device circuit is provided by the voltage formers, which there are five in circuit and they are separated for their intended purpose. Analog voltage supply 250 is designed to power 224* the entire analog part of the circuit, there is also a digital voltage supply 244 providing power 236* to the microcontroller. Power supply for barometer and unit of accelerometer, gyroscope and magnetometer provided 223* by sensors voltage supply unit 249. To supply the LCD screen and the wireless communication unit there are provided two separate voltage supply units 242 (235*) and 243 (234*), respectively. Such a number of voltage supplies is provided in order to separate the above-described units, as well as to reduce interference that they can interfere with each other, and especially on the useful signal from the sensors. The value of the stabilized voltage is Ustab=3.3V.

To charge the battery from an external USB adapter (5V), a charge block 239 is connected to the USB connector 240 mounted on the printed circuit board (PCB) of the device. The battery charge unit is equipped with a red LED 241 to indicate the battery charging process, which turns off when the charging process ends. The power supervisor 248 is a tracking microcontroller, which serves to control the power of the Wireless communication module, initial turn on/off of the device, with single the user button #3 247 press for a long time, the device wakes up by the single brief press of the button, the device enters the firmware update mode, and the reset of device parameters. The processes of switching on and restarting (after a single click of the button 247) are signaled by means of a short vibration using a vibro motor.

The Operation Algorithm of Microcontroller

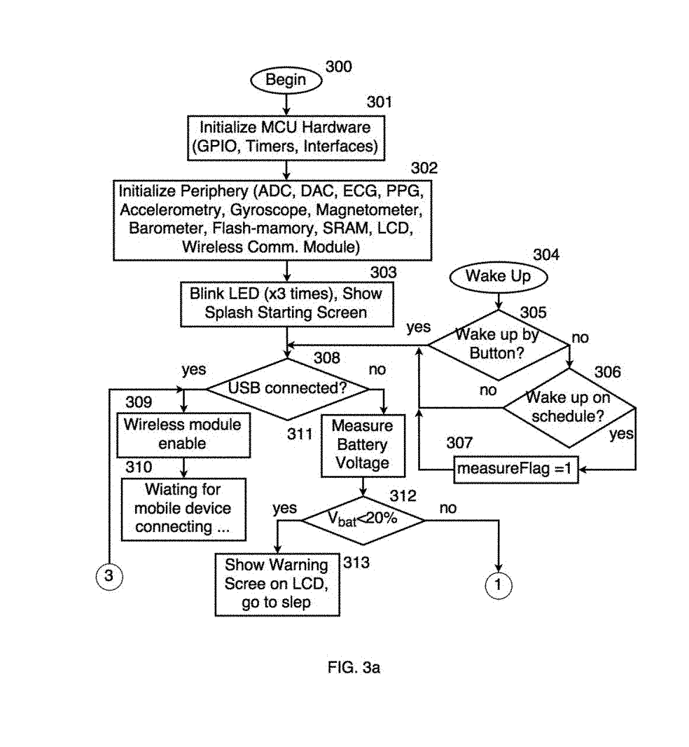

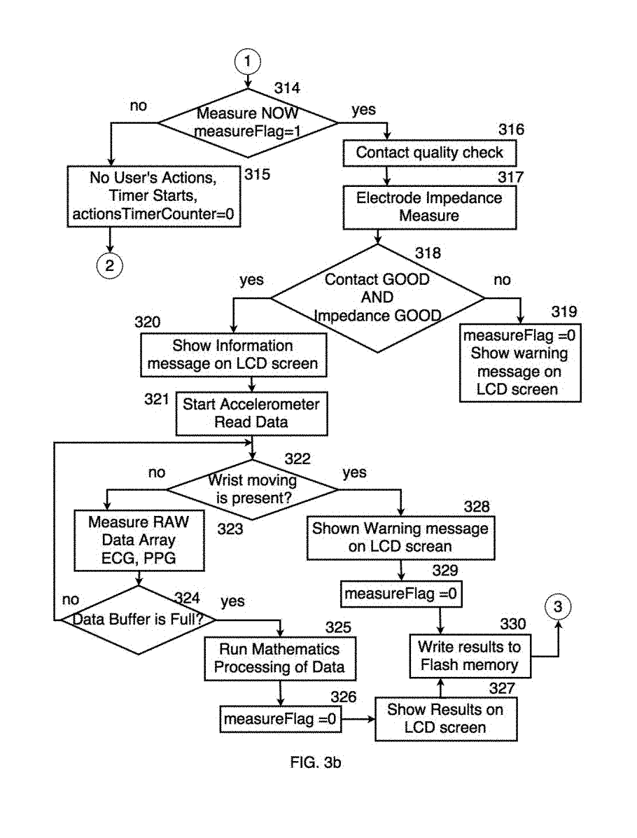

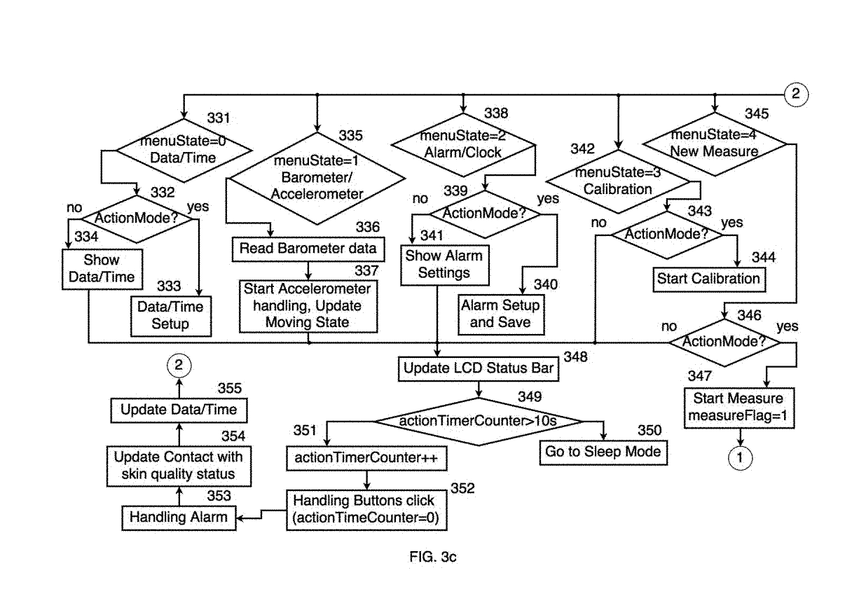

The microcontroller unit (MCU) is used to control the periphery, operating modes of the device, recording signals from sensors and calculating output parameters (blood pressure, etc.), as well as communication with an external mobile device. Schematic representation of the microcontroller algorithm is shown in FIG. 3a, 3b, 3c. The microcontroller algorithm described herein is given for understanding the operating principle of the device and is not limited by this embodiment.

When the power turns on, the device starts 300 and executes the main program code stored in the non-volatile FLASH memory of the microcontroller (MCU). At the first stage, the main microcontroller initializes the hardware 301: the input/output data ports (GPIO), timers and interfaces. After this the initialization of the entire periphery follows 302: analog-to-digital (ADC) and digital to analog converters (DAC), PPG and ECG signals' sensors, accelerometer, gyroscope, magnetometer, barometer, Flash and SRAM memory units, LCD screen and a wireless communication module.

Upon successful completion of the first phase started initial LED-indication 303—3 short blinks of red LED and LCD screen shows splash starting screen. If the device is not initially turned on, but it's “waking up” 304 from the sleep state (waiting mode), then the clause of “wake up” begins to be checked. If this happens by pressing button 305, the device immediately proceeds to the next step. And if the “wakening up” occurred by a pre-set schedule 306, then it means that after that the process of pressure measurement should start, and for this purpose it is necessary to assign a readiness flag to start measurements=1 (307).

The presence of memory in the device circuit allows it to work completely autonomously (without connecting to other external devices for data synchronization) for a while. Therefore, for convenience, reliable transmission of data and reducing power consumption, the function of connecting the device to an external mobile device is carried out only during the charging process of the device. For this purpose, immediately after turning on the algorithm of the microcontroller proceeds the step of checking whether the device is connected to the charger 308 (an external USB adapter). If the USB adapter is connected, the wireless communication unit 309 is enabled and the device enters the standby mode of connection to the mobile device 310. If the external USB adapter is not connected, the battery voltage level 311 is checked before starting measurements. In case when the device is discharged, i.e. the battery voltage level is below 20% (312), the warning message 313 is displayed on LCD screen and the device enters sleep mode waiting for the charge. If the device is charged, then it is ready to work.

First of all, the status of the readiness to start measurements flag is checked 314 at this stage: if measure Flag=1, the microcontroller algorithm goes to the measurement branch, if measure Flag=0 or is not defined, then the items selection of the modes menu is started. When the device is in the state of menu items selection and the user does not perform any actions, the actions Timer Counter timer 315 is turned on, which, after 10 seconds, if there is no action from the user, puts the device into sleep mode to reduce power consumption.

Before the measurements themselves, the device checks the quality of electrodes contact to the human skin 316 and measures the electrode impedance value 317. If the contact and the impedance value do not satisfy the specified parameters 318, it means that the device is not ready for measurements (measure Flag=0) 319 and the user sees a warning message on the screen about the need to correct the device position, otherwise the device is ready to start measurement and displays another warning messages for user 320. This message is necessary in order to warn and remind the user that during the measurement (about 130 seconds) he should not move his hand and be in a calm relaxed state. To track the presence of movements, the device begins 321 to read accelerometer data. If the motion is absent or does not exceed the preset threshold 322, the microcontroller begins to measure the raw ECG and PPG data 323 and write them to the buffer. Until the buffer is full 324, the steps of verifying the presence of movements 322 and recording data 323 are repeated cyclically. When the buffer is full, the program proceeds to the processing of the recorded data and the arterial pressure calculation 325, at the end of which the measurement flag is reset (measure Flag=0) 326, and the measurement results are output to the screen 327 and written to the flash memory of the device 330. If the hand movement exceeding a preset threshold was recorded, the warning message is displayed on screen 328, the measurement flag is reset (measure Flag=0) 329 due to the inability to obtain reliable measurement results at the time of movement, and the information about the wrong (interrupted) measurement is written to device Flash-memory. After that, the device goes to the step of checking the USB connection and the voltage level (308-311).

In this embodiment of the invention, in addition to the main function of measuring blood pressure, there are a number of additional functions, an operating modes menu of the device is provided for selecting them. When readiness flag to measurements measure Flag=0 or is not defined 314, then the device goes to algorithm branch of operating modes menu processing. Depending on the selected menu item (date/time, barometer/accelerometer, alarm clock, calibration or measurement start), the value of the parameter menu State=0 or 1 or 2 or 3 or 4, respectively, which define the way of microcontroller work. If the menu item Time/Date 331 is selected and the “Setup” function 332 is confirmed by simultaneously pressing two buttons 228,229, the device enters the setting mode of the selected parameter 333 (in this case the date and time). If the “setup” function is not selected, the current time and date 334 are displayed on the screen. In the case when the Barometer/Accelerometer menu item 335 is selected, the current value of the atmospheric pressure 336 is displayed on the LCD screen and the accelerometer data is indicated as a flag 337 (presence/absence of user movement). Similar to the Date/Time function, the microcontroller actions are performed when the Alarm menu item 338 is selected: confirming setup 339 the device allows to set the alarm 340, otherwise the pre-set alarm clock 341 is displayed on the screen. To start the calibration process, it is necessary to select the appropriate menu item 342 and confirm action 343 (by pressing two buttons 228,229 at the same time), then the microcontroller will begin the calibration process 344. After selecting the “New Measurement” menu item 345 and confirming the action 346, the program assigns the readiness flag to measurement measure Flag=1 (347) and the process goes to the algorithm branch of the blood pressure measurement (314-330).

While menu items processing, information in status bar 348 is also updated, which includes: contact quality status, alarm status and current time. During the user's work with the menu, the last action timer action Timer Counter works by increasing the value by one every second 351 and passes a check for exceeding the threshold value 349, if the timer value is exceeded 10 seconds, the device enters the sleep mode 350 to save power. While the threshold is not exceeded, the microcontroller monitors the pressing buttons events 352 (user buttons #1 and #2 228,229) and in case of detection one, the device zeroes the time counter action Timer Counter=0. Left button 228 click switches Menu mode state decreasing menu State value. Right button 228 click switches Menu mode state increasing menu State value. Long pressing both buttons simultaneously starts the setup function or performs the selected action, depending on the selected menu item. After that, the microcontroller updates the information for status bar: alarm status 353, contact quality status 354, current time and date 355. After that, the processing of the modes menu goes to the step of checking the USB connection and the battery level.

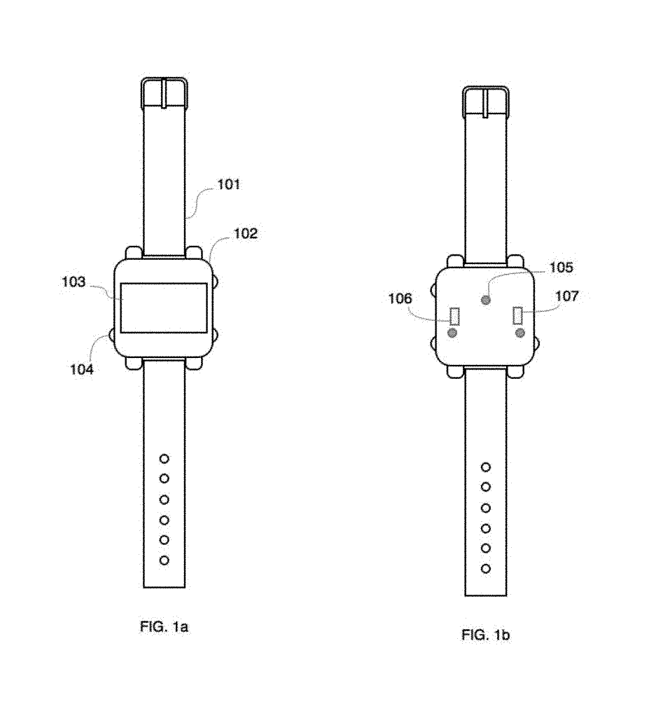

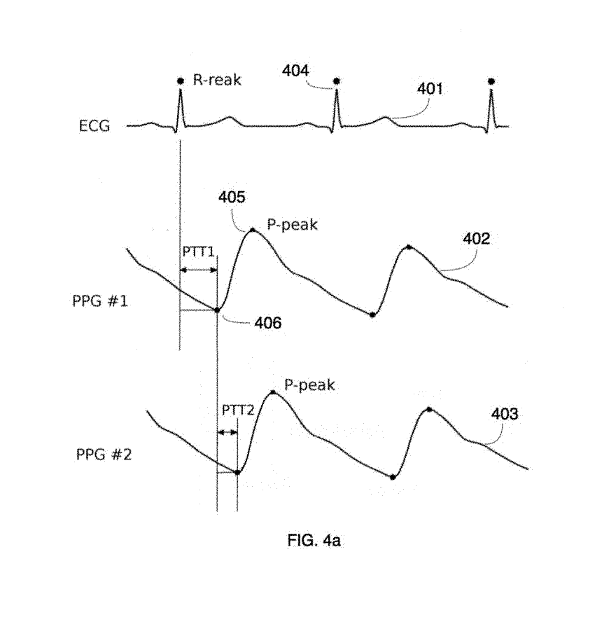

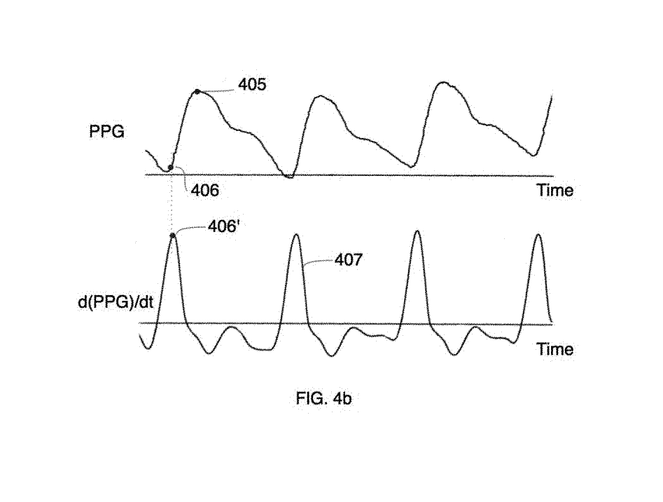

Described herein device uses, as mentioned above, three information signals to calculate the blood pressure. FIG. 4a shows the waveforms and time dependence of all these signals: ECG signal 401 recorded from one hand by “dry” electrodes, two PPG signals 402, 403 recorded by PPG sensor #1 106 and PPG sensor #2 107 correspondingly. The most high-amplitude R-peaks 404 are allocated from the ECG signal, which correspond to the moment of cardiac ventricular depolarization and indirectly indicates the moment of cardiac contraction and the beginning of the subsequent pressure wave. The PPG signal represent the degree of blood filling of the vessels, that is, the P-peak 405 of the PPG signal amplitude corresponds to the moment when the blood volume in the vessel is maximal and does not coincide with the pressure pulse. The moment when the leading edge (beginning) of the pressure pulse reaches the measurement point signifies the beginning the blood volume increasing 406 at the measurement point, which corresponds to the inflection point of the PPG signal curve. This point on the curve can be found by differentiating the signal with respect to time, corresponding P′-peak 406′ on first derivation of PPG signal 407 (see FIG. 4b). For further calculations of blood pressure, the pulse transition time values from the heart to the first PPG sensor (the difference between the corresponding ECG peaks and first derivation of PPG signals in the time scale) and between the two PPG sensors are calculated. The pulse wave velocity in these sections is defined as the distance passed by the pulse divided by the time. An approximate value of length is used to calculate the pulse wave velocity from the heart to the first PPG sensor, it is measured by user as the arm length from the shoulder to the wrist (place where device is fixed) and entered to device as input data before the calibration begins. It is necessary because this distance is individual for each person and taking into account this fact makes it possible to achieve more accurate measurements of blood pressure, otherwise it is taken average value. The distance between the two PPG sensors is constant and is determined by the design of the device (as it shown on FIG. 1b).

Calibration Algorithm

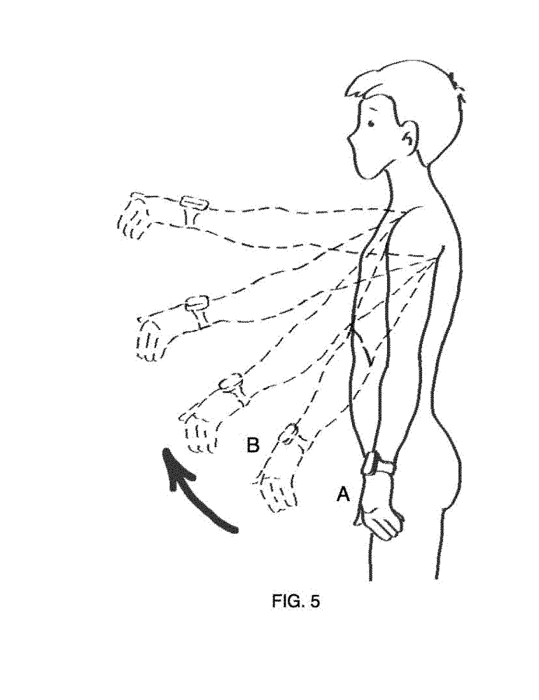

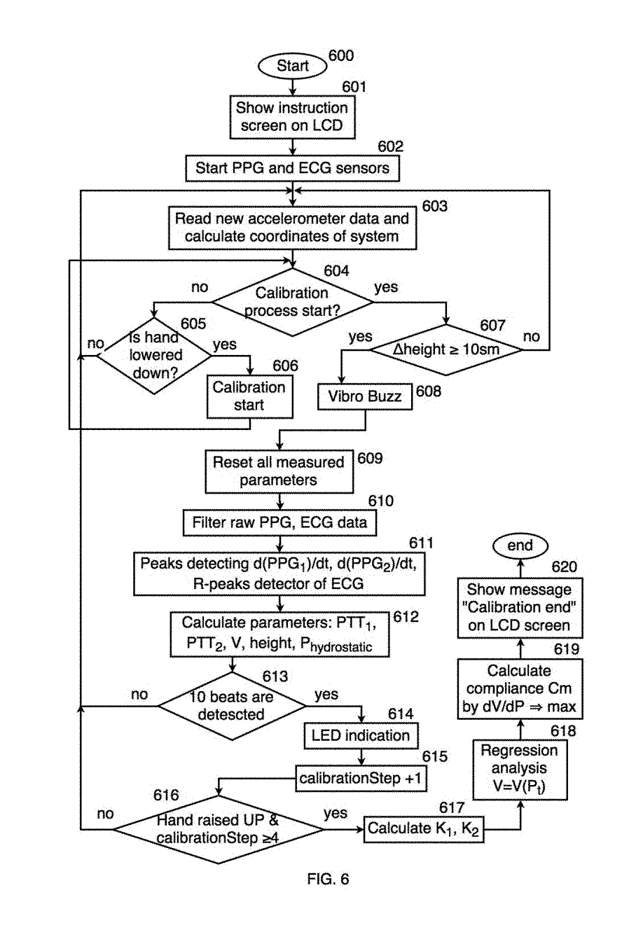

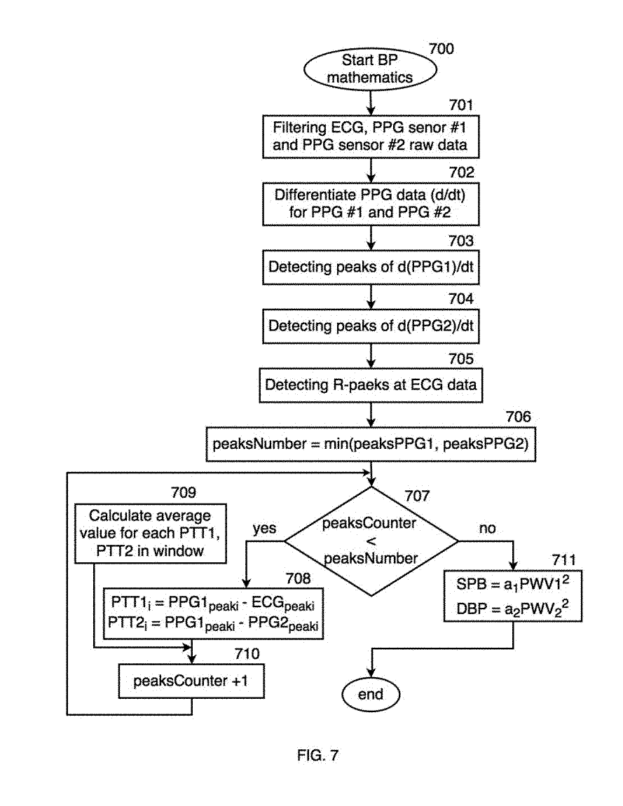

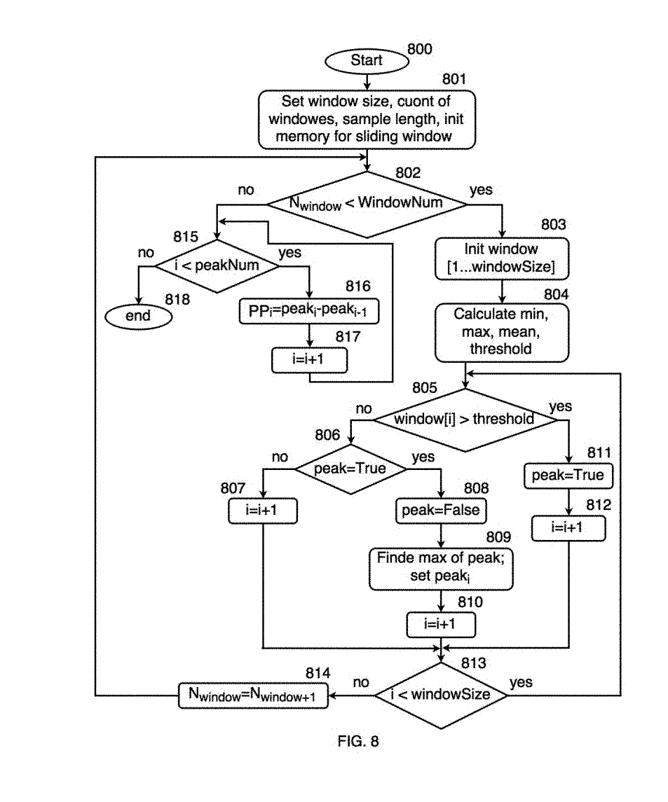

For carrying out calibration measurements, in contrast to the nearest analogue ViSi Mobile by Sotera Wireless, this device does not use a compression cuff. To calibrate the device a gradual raising of the hand is required from the user in such a way as it shown in FIG. 5. The user must perform this action according to the instructions of the device, because the correctness of the calibration directly affects to the accuracy of the further blood pressure measurement, exactly as it described below. After selecting the menu item “Calibration” and confirming it, user should lower the hand down, if there was a vibrating signal, then the calibration process started, fix the hand in this position (about 10 s) and wait for the led indication, then slow raise the hand until the next vibration signal, fix the hand in this position and wait for the light signal, gradually lifting the hand repeat these actions until the device notifies the completion of the calibration (by the corresponding message on the screen).

The calibration algorithm contains the following steps. The calibration algorithm 600 starts after selecting and confirming (by pressing two buttons simultaneously) the corresponding menu item of the device. The LCD screen displays instructions for the user 601, which should be followed to perform the calibration measurements. Then the device starts the PPG and ECG 602 sensors, and begins reading the data from the accelerometer 603 to calculate the system coordinates, which is necessary to determine the position of the user's hand. Thereafter, the device enters 604 a calibration measurement cycle. Before starting the measurement, the device checks the position of the hand 605, if it is lowered, then the calibration measurement process 606 starts, if the hand is not yet lowered, then the device read the data from the accelerometer and wait till the user drops his hand down. At the beginning of each stage of the cyclic measurement, the device verifies the movements of the arm 607 relative to its position during the previous measurement, position A and B in FIG. 5, it should exceed 10 cm (for the first position, when the hand is at the bottom, the initial point is assumed to be 0, so Δheight is always greater than 10 cm). Once this threshold is exceeded, the device vibrates 608, to make it clear for user that it is necessary to stop lifting the hand and fix it in this position for the next measurement step. The values measured in the previous measurement are reset 609.

Signals from both PPG and ECG sensors are filtered 610 in order to eliminate interference. After that, 3 detectors operate in parallel on three signals 611: the R-peaks detector of the ECG signal and two detectors operating with differentiated signals of PPG₁ and PPG₂ and allocating wave peaks (because the differentiated signals are better correlated with the pressure values than the PPG signals themselves). During the measurement, the following parameters 612 are calculated: the mean pulse transition time from the heart to the wrist PTT₁ and between the two PPG sensors PTT₂, the volume of blood circulating at measurement site V, the height of the device, the hydrostatic blood pressure of H_{ydrostatic}. In each position of the hand, the device carries out a measurement until it registers 10 beats of pulse 613, the necessary minimum to carrying out reliable calculations. As soon as the device collects all the necessary data, it is ready to proceed to the next stage of the cyclic calibration measurement 615 and for this purpose the user is given a light signal 614 indicating the necessity to continue raising the hand. If the user's hand is lifted up and there were at least 4 or more calibration measurements 616, than the device proceeds to calculate the parameters K1 and K2 617, which determine the individual user parameters such as blood density, radius and elasticity of the vessels, etc., which affect on blood pressure values. Regression analysis 618 is performed to find the circulating blood volume as a function of the transmural pressure V=V(Pt). On the basis of the obtained data, the value of the vessels compliance 619 is calculated as the maximum of the function dV/dP. This completes the calibration process, the message “Calibration is over” is displayed for the user on the LCD screen 620. All data received during the calibration process is stored in the device memory and then used to calculate blood pressure.

Blood Pressure Measurement Algorithm

When the data buffer 324 is filled during data recording from the ECG and PPG sensors, the device proceeds directly to the blood pressure value measurement from the data written into buffer. After starting the calculation process 700, “raw” data from the ECG, PPG #1 and PPG #2 sensors are subjected to additional digital filtering 701 to reduce the interference level, which can subsequently lead to erroneous detection of peaks. After filtering, each of the two PPG signals are differentiated with respect to time 702, this is necessary, as mentioned above, to determine the points on the curve corresponding to the leading edge of the pressure pulse. Further in the pressure measurement algorithm, only the differentials of the PPG signals are involved, and not they themselves. In the next step, the detectors 703 and 704 come into operation, the operation algorithm of which will be described below. They determine the peaks on the curves of the differentiated signals of the PPG #1 and PPG #2 sensors. These detectors work in parallel with the detector of the R-peaks of the ECG signal 705. Since this device uses the principle of recording the ECG signal described in the pending patent application entitled: THE METHOD OF REGISTERING THE INTERVALS BETWEEN ADJACENT R-PEAKS OF ECG SIGNAL WITH ONE HAND IN ORDER TO DIAGNOSE AND ASSESS THE STATE OF THE HUMAN BODY AND HEART RATE VARIABILITY WEARABLE MONITORING DEVICE (U.S. Ser. No. 15/442,631, filed Mar. 18, 2017), the detailed principle of R-peak detector operation is described in the same source. Briefly, the detector operates on the principle of a sliding window for which a threshold value is continuously calculated, in the event of exceeding which the R-peak is detected on the ECG signal curve.

Then the total number of peaks (peaks Number) in recorded signals is counted (706) as the minimum value of their total numbers detected from PPG #1 and PPG #2 separately. Next, by parallel scanning on all three signals from the first to the last peak 707 (by means of a dynamic window in the peak-to-peak mode 710), the pressure pulse transition time values are calculated 708 for two sections of pathway: from the heart to the first PPG sensor (PTT 1) and between two PPG sensors (PTT 2). Then average values of PTT for each data set from PTT #1 and PTT #2 are calculated. When data scanning process reaches the ends of each recorded signals, algorithm of microcontroller goes right to systolic and diastolic blood pressure calculation 711 from average values of PTT1 and PTT2. As many research show and experimentally established that systolic blood pressure value better correlates with PTT1 (the pulse transition time from heart to the wrist) and in its turn the diastolic pressure value correlates with PTT2. Exactly of this reason, described herein device uses such kit of sensors and the systolic pressure is calculated from the value of PWV 1 obtained from the averaged PTT 1, and the diastolic from PWV 2, respectively. At this point, the measurement process ends.

Operation Algorithm of Peaks Detector on d(PPG)/dt

Immediately after differentiating the PPG signal, the P′-peak and PP intervals detector starts 800. P′-peak is a peak on the curve of the differentiated PPG signal (d(PPG)/dt) that correspond to the inflection point (point of the fastest increment) of the PPG curve and indicate the beginning of the pressure pulse wave. The size of the sliding window, the number of splits of the signal sample are computed, and the memory for the sliding window buffer is initialized 801. A window counter that determines the number of the current window starts and changes from 1 to the number of the sample partitions 802, 803. Statistical parameters such as the maximum, minimum, average value and the value of the dynamic threshold level of the signal within this window are calculated 804 for current data in the sliding window buffer. Exceeding the value of amplitude threshold is checked for each signal value in the window buffer 805. If the signal did not exceed the threshold value and before it was not fixed peak 806, the processing of the next signal fragment 807 continues. If the peak was detected and the signal level passed below the threshold level, the peak endings are recorded and the peak flag is reset 808, an index corresponding to the maximum value of the signal amplitude during the peak fragment is found 809, 810. The index of found peak is written to the global peak array 813, 814. If the threshold has been exceeded, the flag of peak fragment detection is set as TRUE 811, 812.

After the end of the search of all window intervals 802, the values of PP intervals are calculated, as the difference between the indexes of adjacent peaks 815, 816, 817, knowing the sampling frequency, the values are transferred to time units of measurement (ms) and written to the global array of PP intervals, for further work with them 818. To work with three signals simultaneously, specifically for calculation of PPT for different signal combinations, the index of each peak on all three signals is used, as a timestamp for the same pressure pulse.

Mathematical Foundation of Blood Pressure Calculation

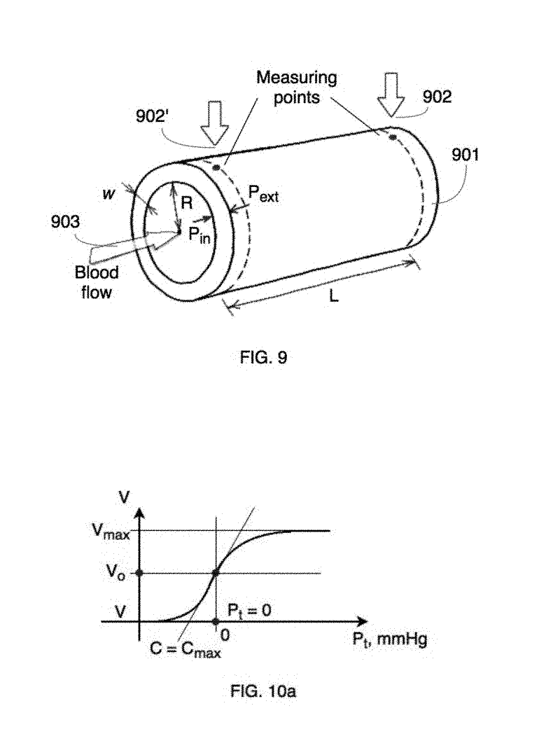

For a better understanding of the method of calculating blood pressure, as well as for a visual explanation of some of the values used for this purpose, FIG. 9 is shown, which schematically shows some arbitrary section of the vessel 901 on which measurements are made with indicating the points of measurement 902, 902′ and the arrow of the blood flow direction 903. The following symbols are shown in the figure: R is the radius of the vessel, to simplify the calculation, we take the section on which the vessel has the constant radius, but this parameter will vary from person to person; W is the thickness of the vessel wall; Pᵢₙ and Pₑₓₜ—internal and external pressure, respectively, directed perpendicular to the vessel's walls and opposite to each other; L is the length of the portion of the vessel on which the measurement is made.

PWV, the velocity at which the arterial pulse propagates through the artery tree, can be used as a measure of arterial stiffness. It has a strong correlation with cardiovascular events and all-cause mortality, and was recognized by the European Society of Hypertension, as a useful additional test in the investigation of hypertension (thanks to correlation between PWV and blood pressure). A high pulse wave velocity (PWV) has also been associated with poor lung function.

The relationship between pulse wave velocity (PWV) and artery wall stiffness (expressed as the incremental elasticity modules of the artery) is shown in Moens-Korteweg equation, which is derived from Newton's second law of motion using some simplifying assumption and has the form of Equation 1:

$$PWV = \sqrt{\frac{E \cdot w}{2 R \cdot ρ}} , \qquad ( 1 )$$

where E is the incremental elastic modulus of the vessel wall or its distensibility; ρ is density of blood. All parameters in right part of this equation do not change fast for the same human, but different from person to person. That is why calibration measurement is necessary to take in account influencing of individual users' parameters on calculation of blood pressure value. The Moens-Korteweg equation (1) states that PWV is proportional to the square root of the incremental modulus of the vessel wall at a constant ratio of wall thickness W to the vessel radius R. On the other side PWV can be expressed as the ratio of the path of pressure pulse wave L to the pulse transition time PTT of this path in form of Equation 2 below:

$$PWV = \frac{L}{PTT} . \qquad ( 2 )$$

Using the Hooke's law, the incremental modulus of elasticity E can be expressed by means of the Equation 3 below:

E=E_{O}·e^{ξP}^{ₜ} (3),

where E_{O} is the Young's modulus; Pₜ is the transmural pressure; ξ is a coefficient. Transmural pressure, Pₜ is the difference between blood pressure in the cavity of the blood vessel, that is, between intracavitary (intraluminal) pressure and extra-cavity (interstitial) pressure (eg, applied external pressure or tissue pressure surrounding the vascular cavity). Essentially, the transmural pressure is equivalent, identical to the pressure of filling the blood in the cavity of the vessel. The gradient of transmural pressure is directed perpendicular to the wall of the vessel. Transmural pressure can be described by help of Equation 4:

Pₜ=Pᵢₙ−Pₑₓₜ±ρgh (4),

where Pᵢₙ is the internal pressure in the vessel, in fact it is the same pressure created by blood P_{BP}; Pₑₓₜ—external pressure applied to the vessels' walls by surrounding tissues (including external pressure applied to the extremity at the measurement point, if one takes place); ρgh—hydrostatic blood pressure (+ or − depends on the position of the limb).

Taking into account the Equation 4, the square of the pulse wave velocity can be written as Equation 5:

$${PWV}^{2} = \frac{{E}_{o} \cdot w}{2 ρ R} \cdot {e}^{ξ {P}_{t}} . \qquad ( 5 )$$

Now, for simplicity, it is possible to introduce coefficients reflecting the effect of individual user parameters on the relationship between PWV and transmural pressure. If we take the coefficients equal to the following expressions

$${K}_{1} = \frac{{E}_{o} \cdot w}{2 ρ R} ;$$

K₂=ξ, then Equation 5 acquires the following simplified form, as Equation 6 shown below:

PWV²=K₁·e^{K}^{₂}^{P}^{ₜ} [symbol] (6).

Pulse wave velocity PWV varies with the blood pressure. This is clearly seen from the Bromwell-Hill equation, which relates the pulse wave velocity to the characteristics of the vessel and fluid (in our case, blood) and has the form Equation 7:

$$PWV = \frac{1}{\sqrt{ρ \cdot Distensibility}} . \qquad ( 7 )$$

This equation assumes that the vessel is compliant and fluid which fills it is incompressible and nonviscous. The Distensibility of an infinitesimally thin slice of a vessel under fluid pressure is calculated as follows

$$Distensibility = \frac{strain}{Δ P} = \frac{Δ S}{S \cdot Δ P} \qquad ( 7.1 )$$

and can be expressed through the compliance of the vessel. The compliance is the ability of a blood vessel wall to expand and contract passively with changes in pressure which is an important function of large arteries and veins. This capacity of the vessel to expand and increase with increasing transmural pressure is quantitatively defined as the compliance (C) and is expressed through the volume change (ΔV) divided by the pressure change (ΔP) and shown as Equation 8:

$$C = \frac{Δ V}{Δ P} . \qquad ( 8 )$$

The pulse wave velocity according to Bromwell-Hill expressed through the compliance has the form of Equation 9:

$$PWV = \sqrt{\frac{V \cdot Δ P}{ρ \cdot Δ V}} , \qquad ( 9 )$$

for convenience of calculation the square of the velocity is used in further calculation and shown as Equation 9.1:

$${PWV}^{2} = \frac{V}{ρ C} . \qquad ( 9.1 )$$

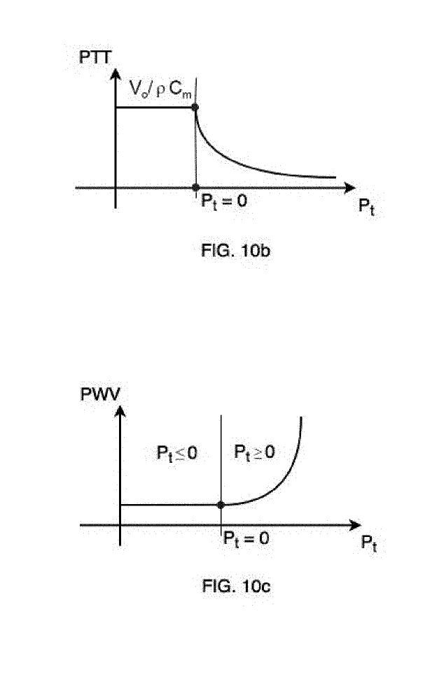

The dependence curve of the volume on the pressure has the form of a sigmoid and is shown in FIG. 10a. In the case when the value of transmural pressure is zero (Pₜ=0), a volume of blood Vₒ passes through the vessel, it is possible when the influence of internal and external pressure equalize each other. Since no additional external pressure is applied to the user's limb during measurement, it can be assumed that only the influence of the environment is acting as an external influence. The atmospheric pressure value is monitored with a barometer to ensure that the external pressure is not changed during the measurement. In such a case, reaching the point at which the transmural pressure is zero is possible due to a change in the magnitude and “sign” (+ or −) of the hydrostatic blood pressure. This point is marked on the curve of FIG. 10a and is the main one for the subsequent calculations, in it C tends to the maximum value C=Cₘₐₓ further mentioned as (Cₘ). If the graph of the dependence of volume on pressure is conditionally divided into two parts at the point where Pₜ is zero, the volume can be written in the system of Equations 10:

$$V = {V}_{o} \cdot {e}^{\frac{{C}_{m}}{{V}_{o}} {P}_{t}} , {P}_{t} < 0 ; \\ V = {V}_{m} - ( {V}_{m} - {V}_{o} ) \cdot {e}^{\frac{- {C}_{m}}{( {V}_{m} - {V}_{o} )} {P}_{t}} , {P}_{t} \geq 0. \qquad ( 10 )$$

Taking the derivative dV/dP along the curve in FIG. 10, obtain the compliance of the vessels

$$C = \frac{dV}{dP} .$$

It can also be described using the system of Equations 11 below:

$$C = {C}_{m} \cdot {e}^{\frac{{C}_{m}}{{V}_{o}} {P}_{t}} , {P}_{t} < 0 ; \\ C = {C}_{m} \cdot {e}^{- \frac{{C}_{m}}{{V}_{m} - {V}_{o}} {P}_{t}} , {P}_{t} \geq 0. \qquad ( 11 )$$

Further, substituting the resulting system of Equations 11 in the above-mentioned Equation 9.1, we obtain a new system of Equations 12 shown below, which describes the dependence of the pulse wave velocity on the vessel's compliance (C) and transmural pressure (Pₜ).

$${PWV}^{2} = \frac{{V}_{o}}{ρ {C}_{m}} , {P}_{t} < 0 ; \\ {PWV}^{2} = \frac{{V}_{m}}{ρ {C}_{m} \cdot {e}^{- \frac{{C}_{m}}{{V}_{m} - {V}_{o}} {P}_{t}}} , {P}_{t} \geq 0. \qquad ( 12 )$$

As can be seen from the system of Equations 12, in the case when Pₜ<0, the pulse wave velocity does not depend on the transmural pressure value, provided that Cₘ is maximal and does not change. That is

$$\frac{{V}_{o}}{ρ {C}_{m}} = const$$

and this section on the dependence curve of PWV on Pₜ shown in FIG. 10c has the form of a straight line. When passing through the point Pₜ=0, the function increases. As practice shows, the first part of the function, in which Pₜ<0, corresponds to the process of raising the hand, as shown in FIG. 5, up to a certain level at which Pₜ=0. Exactly for the determination of the point at which the external and internal pressures balance each other, the calibration process is carried out in the form of a gradual raising of the hand with short measurements every 5-10 cm. Such measurements are made in fixed definite distance, determined by means of an accelerometer, in order that in the future it would be possible to calculate the value of the hydrostatic blood pressure, since it has a contribution to the value of transmural pressure (see Equation 4). FIG. 10b shows the dependence of PTT on transmural pressure. Since PTT is the inverse value of PWV, as it shown in Equation 2, the shape of the curve in FIG. 10b is also the inverse of the function in FIG. 10c.

Searching the point where the transmural pressure is zero necessary for calculation the first coefficient of individual user's parameters K₁. If Pₜ=0, then Equation 4 can be transformed in such a way as to express blood pressure as it is shown in Equation 13:

P_{BP}=Pₑₓₜ±ρgh. (13)

Since in the case when Pₜ=0, then e^{K}^{₂}^{P}^{ₜ}=1 so Equation 6 taking into account the Equation 2 acquires the form shown as Equation 14 below, which allows to calculate K₁:

$${K}_{1} = {PWV}_{o}^{2} = \frac{{L}^{2}}{{PTT}_{o}^{2}} \begin{matrix} \\ \end{matrix} . \qquad ( 14 )$$

There PWVₒ and PTTₒ are pressure pulse wave velocity and pulse transition time correspondingly at the posture where transmural pressure is zero (Pₜ=0).

To find the second coefficient K₂, it is necessary to perform measurements at two adjacent positions, for example, positions A and B in FIG. 5, in which Pₜ≠0 and h≠0. These two positions can be described by the system of Equations 15 shown below, since two measurements are carried out in a short time and with a small interval, it can be assumed that in these states any parameters except height does not change or their variation is so insignificant that they can be neglected (ignored).

PWV₁²=K₁·e^{K}^{₂}^{(P}^{_{BP}}^{±ρgh}^{₁}^{-P}^{ₑₓₜ}⁾

PWV₂²=K₁·e^{K}^{₂}^{(P}^{_{BP}}^{±ρgh}^{₂}^{-P}^{ₑₓₜ}⁾ (15)

If now the first equation from the system of equations 15 divide into the second, we obtain the Equation 16 from which it is easy to find what the second coefficient K₂ will be equal to.

$$\frac{{PWV}_{1}^{2}}{{PWV}_{2}^{2}} = {e}^{{K}_{2} [ ρ ℊ ( {h}_{1} - {h}_{2} ) ]} = {e}^{{K}_{2} ρ ℊΔ h} \qquad ( 16 )$$

The coefficient K₂ can be calculated from Equation 17 below:

$${K}_{2} = \frac{2}{ρ ℊΔ h} ln ( \frac{{PTT}_{2}}{{PTT}_{1}} ) \qquad ( 17 )$$

Transmural pressure substantially is the arterial pressure that is measured in clinical settings using Korotkoff's method. If express it through the coefficients K1 and K2 obtained in the course of the calculations, it can be described by Equation 18:

$${K}_{2} \cdot {P}_{t} = ln \frac{{PWV}^{2}}{{K}_{1}} \\ {P}_{t} = \frac{1}{{K}_{2}} ln \frac{{PWV}^{2}}{{K}_{1}} \\ {P}_{t} = {K}_{2}^{′} ln {K}_{1}^{′} {PWV}^{2} \qquad ( 18 )$$

For convenience of further calculations, the coefficients K₁′ and K₂′ have been introduced, which are inverse values of K₁ and K₂, respectively (Equations 19).

$${K}_{1}^{′} = \frac{1}{{K}_{1}} ; {K}_{2}^{′} = \frac{1}{{K}_{2}} \qquad ( 19 )$$

This is necessary, since PWV which is used in pressure calculations, is the inverse of PTT (see Equation 2). As it mentioned above the systolic blood pressure (SBP) are better correlated with the PWV and PTT values obtained from the ECG and PPG #1 signals, that is calculated for path from the heart to the wrist, so PWV_{I} is used for calculation SBP. And to calculate the diastolic blood pressure (DBP), the data (PWV_{II}) from the PPG #1 and PPG #2 sensors are used. The final system of Equations 20 for the calculation of systolic and diastolic blood pressure is shown below:

SBP=K′₂·ln K′₁PWV_{I}²

DBP=K′₂·ln K′₁PWV_{II}² (20)

As it seen from all calculation described above this invention allows to calculate systolic and diastolic blood pressure without using compression cuff only due to hydrostatical property of liquids one of which blood is. It allows to avoid the number of problems associated with use of compression cuff, which was already mentioned.

1. A method of blood pressure monitoring by a recording device designed in one housing, with bottom and top surfaces, and fixed to a wrist by means of fixation elements, contact electrodes are located on the bottom surface of the housing, the housing contain a set of electronic and optical sensors and an electronic circuit for sensors signals processing,
the method comprises the operations:
checking a contact quality of electrodes, which are used for recording an electrical activity of a human heart (hereinafter, ECG electrodes), with a skin by measuring contact resistance and comparing obtained data with tabulated values,
parallel recording of samples, as arrays of potential difference instantaneous values at a output of each of the sensors, for all three information signals: the electrical activity signal of the heart (hereinafter ECG) and signals from optical sensors (PPG) reflecting a blood vessel filling process,
detection points on the ECG signal which satisfy R-peaks signs (the highest amplitude peak of the ECG signal, hereinafter R-peaks) and writing indices into R′-peaks array, in the form of time marks for each peak corresponding to a peak moment,
calculating R-R-intervals values as a time delays in msec between two subsequent R-peaks and writing to the R-R intervals array,
obtaining a dependence of the vascular blood flow rate on time by differentiating the optical sensors signals (hereinafter PPG #1 and PPG #2, correspondingly) in order to detect the peaks P1′ and P2′ (respectively for derivations of signals PPG #1 and PPG #2) corresponding to the maximum rate of blood volume increasing in underlying vessels and indicating a time point when the pressure pulse propagating from the heart reaches the location point of the corresponding sensor;
recording indices as the timestamps for each P1′ and P2′-peaks;
determination of a pressure pulse transition time (further PTT) on two segments: a first—from the heart to the wrist, a second—between the two optical, sensors on the wrist by calculating the time delay PTT1 between the corresponding R-peaks on the ECG signal and the P1′-peaks on the derivation of signal PPG #1, for the first segment, and the PTT2 delay between the respective P1′ and P2′-peaks for the derivation of PPG #1 and PPG #2 signals as the difference between the time indices of the detected peaks;
determination of systolic pressure from the average PTT1 value and diastolic blood pressure from the averaged PTT2 values, taking into account the individual coefficients reflecting the effect of individual user parameters on the calculated pressure values

2. The method of blood pressure monitoring of claim 1, wherein the tabulated values of contact resistance used for comparing and determining the quality of electrodes' contact with the user's skin are accepted standard from the reference literature

3. The method of blood pressure monitoring of claim 1, wherein the values of the individual coefficients for the blood pressure calculation are obtained by pre-calibrating the device for each user

4. The method of blood pressure monitoring of claim 1, wherein the values of the individual coefficients for the blood pressure calculation are obtained by entering values of individual parameters of this user manually, as inputs for the pressure calculation.

5. The method of blood pressure monitoring of claim 3, wherein to obtain accurate blood pressure values, the pre-calibration of the device is performed by the same device by a series of measurements for different positions of the user's hand at different heights

6. The method of blood pressure monitoring of claim 3, wherein the calibration process for determining individual coefficients is performed by measuring a pulse wave velocity (PWV) in several positions of the user's hand at different heights in order to determine a dependence of the individual parameters on the pressure change that is provided by the change of a hydrostatic component of the blood pressure,
the method comprises the operations:
sequential measurement of the pulse wave velocity in different positions of the user's hand (at different heights),
determination of the position of the user's hand where the external and internal pressure on the vessels' walls balance each other by means of changing the hydrostatic component of the blood pressure
determination of the individual coefficients

7. The method of blood pressure monitoring of claim 4, wherein the values of the individual coefficients for calculating blood pressure are obtained from the numerical values of the individual user parameters measured in a number of laboratory studies and entered manually into the device as inputs to the determination of blood pressure.

8. The method of blood pressure monitoring of claim 7, wherein the process of determining the individual user coefficients from the numerical values of the individual user parameters is used instead of carrying, out pre-calibration measurements, as an alternative method.

9. (canceled)

10. (canceled)

11. The device for blood pressure monitoring of claim 10, wherein two optical sensors (hereinafter photodetectors) are made in the form of a combination of a photo-emitter and a photodetector

12. (canceled)

13. (canceled)

14. (canceled)

15. (canceled)
